(19) United States
(12) Patent Application Publication
(10) Pub. No.: US 2013/0072440 A1
(43) Pub. Date: Mar. 21, 2013

(54) CO-CRYSTALS AND PHARMACEUTICAL FORMULATIONS COMPRISING THE SAME

(71) Applicants: Eleni Dokou, Cambridge, MA (US); Dragutin Knezic, Watertown, MA (US)

(72) Inventors: Eleni Dokou, Cambridge, MA (US); Dragutin Knezic, Watertown, MA (US)

(73) Assignee: VERTEX PHARMACEUTICALS INCORPORATED, Cambridge, MA (US)

(21) Appl. No.: 13/608,079

(22) Filed: Sep. 10, 2012

Related U.S. Application Data
(63) Continuation of application No. PCT/US2011/028081, filed on Mar. 11, 2011.
(60) Provisional application No. 61/312,841, filed on Mar. 11, 2010.

(51) Int. Cl. A61K 38/07

(52) U.S. Cl. 514/20.1; 514/21.9

(57) ABSTRACT

The pharmaceutical composition including a co-crystal. The co-crystal includes a) a co-former; and b) an active pharmaceutical ingredient (API) with solubility in water is less than one part by weight of the API in ten parts by weight of water. Furthermore, the formulation includes a polymer, and a weight ratio of the co-crystal to the polymer is about 0.5:99.5 to about 99.5:0.5. The kinetic solubility of the co-crystal after being in contact with an environment of use is at a therapeutically acceptable level for a prolonged period of time.

### CROSS REFERENCE TO RELATED APPLICATIONS

The present application claims the benefit of PCT application number PCT/US2011/028081, filed Mar. 11, 2011, which claims priority to U.S. Provisional Application No. 61/312,841, filed Mar. 11, 2010. The entire contents of these applications are incorporated herein by reference.

### BACKGROUND OF THE INVENTION

Active pharmaceutical ingredients (API or APIs) in pharmaceutical compositions can be prepared in a variety of different forms. Such APIs can be prepared so as to have a variety of different chemical forms including chemical derivatives or salts. Such APIs can also be prepared to have different physical forms. For example, the APIs may be amorphous, may have different crystalline polymorphs, or may exist in different solvation or hydration states. By varying the form of an API, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapour pressure, density, color, and compressibility. Accordingly, variation of the crystalline state of an API is one of many ways in which to modulate the physical properties thereof.

It would be advantageous to have new forms of these APIs that have improved properties. Specifically, it is desirable to identify improved forms of APIs that exhibit significantly improved properties including increased aqueous solubility and stability. For example, formulating a practically insoluble API in water, to be suitable for administration is difficult. Further, it is desirable to improve the processability, or preparation of pharmaceutical formulations. It may be also desirable to increase the dissolution rate of API-containing pharmaceutical compositions, increase the bioavailability of orally-administered compositions, and provide a more rapid onset to therapeutic effect. It is also desirable to have a form of the API which, when administered to a subject, reaches a peak plasma level faster, has a longer lasting therapeutic plasma concentration, and higher overall exposure when compared to equivalent amounts of the API in, for example, its crystalline form.

Therefore, there is a need for a pharmaceutical composition that provides an improved formulation including a co-crystal that keeps the poorly soluble API dissolved, instead of crashing out, in an environment of use over a prolonged period of time and that provides therapeutically effective levels of the API concentration in the environment of use.

### SUMMARY OF INVENTION

The present invention is directed to a pharmaceutical formulation comprising a co-crystal.

It is an object of the present invention to provide a formulation comprising a co-crystal of a poorly soluble API that promotes a sufficient level of bioavailability to be therapeutically effective in an environment of use and maintains that level for a prolonged period of time.

In one aspect, the present invention includes a pharmaceutical composition. The pharmaceutical composition includes a co-crystal. The co-crystal includes:
a) a co-former; and
b) an active pharmaceutical ingredient (API) with solubility in water is less than one part by weight of the API in ten parts by weight of water. Furthermore, the formulation includes a polymer, and a weight ratio of the co-crystal to the polymer is about 0.5:99.5 to about 99.5:0.5. The kinetic solubility of the co-crystal after being in contact with an environment of use is at a therapeutically acceptable level for a prolonged period of time.

In certain embodiments, the formulation comprises particles of the co-crystal and the polymer in intimate association with each other.

Typically, the co-crystal is present in an amount about at least 30% of the total weight of the formulation. In one embodiment, the co-crystal is present in an amount about at least 35% of the total weight of the formulation. In another embodiment, the co-crystal is present in an amount about at least 40% of the total weight of the formulation. In yet another embodiment, the crystalline composition is present in an amount about at least 45%, 50%, 55%, 60%, 70% or 80% of the total weight of the formulation.

In certain embodiments, the kinetic solubility of the co-crystal after being in contact with an environment of use for at least two hour is greater than 0.100 mg/ml.

In certain embodiments, the kinetic solubility of the co-crystal after being in contact with an environment of use for at least three hour is greater than 0.100 mg/ml.

In certain embodiments, the pharmaceutical formulation further includes one or more excipients.

In certain embodiments, the one or more excipients is selected from the group consisting of a filler, a surfactant, a glidant, a lubricant and a disintegrant.

In certain embodiments, the one or more excipients includes one or more fillers. Examples of the fillers can include, but are not limited to, the following: mannitol, lactose, sucrose, dextrose, maltodextrin, sorbitol, xylitol, powdered cellulose, microcrystalline cellulose, silicified microcrystalline cellulose, methylcellulose, ethylcellulose, hydroxyethylcellulose, methylhydroxyethylcellulose, talc, starch, pregelatinized starch, dibasic calcium phosphate, calcium sulfate and calcium carbonate. In some embodiments, the one or more fillers is a cellulosic filler. In some embodiments, the one or more fillers is lactose, microcrystalline cellulose or silicified microcrystalline cellulose. In some embodiments, the one or more filler is silicified microcrystalline cellulose.

In certain embodiments, the one or more excipients includes one or more disintegrants. In certain embodiment, the formulation includes about 1 wt. % to about 30 wt. % of the one or more disintegrants. Examples of the disintegrants can include, but are not limited to, the following: croscarmellose sodium, sodium alginate, calcium alginate, alginic acid, starch, pregelatinized starch, sodium starch glycolate, crospovidone, cellulose and its derivatives, carboxymethylcellulose calcium, carboxymethylcellulose sodium, soy polysaccharide, guar gum, an ion exchange resin, an effervescent system based on food acids and an alkaline carbonate component, and sodium bicarbonate. In one embodiment, the one or more disintegrants is croscarmellose sodium.

In certain embodiments, the one or more excipients can include one or more surfactants. In certain embodiments, the formulation includes about 0.1 wt. % to 30 wt. % of the one or more surfactants. Examples of the surfactants may include, but are not limited to the following: sodium lauryl sulfate, docusate sodium, polyoxyethylene sorbitan fatty acid esters, polyoxyethylene 20 stearyl ethers, polyoxyethylene alkyl ethers, polyoxyethylene castor oil derivatives, pegylated hydrogenated castor oils, sorbitan esters of fatty acids, Vitamin E or tocol derivatives, vitamin E TPGS, tocopheryl esters, lecithin, phospholipids and their derivatives, poloxamers, stearic acid, oleic acid, oleic alcohol, cetyl alcohol, mono and diglycerides, propylene glycol esters of fatty acids, glycerol esters of fatty acids, ethylene glycol palmitostearate, polyoxylglycerides, propylene glycol monocaprylate, propylene glycol monolaurate and polyglyceryl oleate. In one embodiment, the one or more surfactants is sodium lauryl sulfate.

In certain embodiments, the one or more excipients can include one or more glidants. Examples of the glidants may include, but are not limited to, talc, colloidal silica (e.g., Cabosil M-5), magnesium oxide, magnesium silicate, leucine and starch. In one embodiment, the one or more glidants is colloidal silica.

In certain embodiments, the one or more excipients can include one or more lubricants. In certain embodiments, the formulation includes about 0.1 wt. % to 30 wt. % of the one or more lubricants. Examples of the lubricants may include, but are not limited to, talc, fatty acid, stearic acid, magnesium stearate, calcium stearate, sodium stearate, glyceryl monostearate, sodium lauryl sulfate, sodium stearyl fumarate, hydrogenated oils, fatty alcohol, fatty acid ester, glyceryl behenate, mineral oil, vegetable oil, leucine, sodium benzoate, or a combination thereof. In another embodiment, the one or more lubricants is sodium stearyl fumarate.

In certain embodiments, the polymer is selected from the following: hydroxypropylmethyl cellulose (HPMC), hydroxypropylmethyl cellulose acetate succinate (HPMCAS), polyvinylpyrrolidone (PVP), polyethylene glycol (PEG), poly(ethylene oxide) (PEO), Polyvinylpyrrolidone-co-vinylacetate (PVPVA), poly(meth)acrylate (e.g., Eudragit® polymers), Polyvinylpyrrolidone (PVP), Polyvinylacetate (PVA) or a combination thereof. In certain embodiment, the polymer is HPMCAS or HPMC. In certain embodiment, the polymer is HPMC.

In one embodiment, the formulation comprises about 5 wt. % to 90 wt. % of the co-crystal, about 5 wt. % to 90 wt. % of a filler, about 1 wt. % to 30 wt. % of the polymer, about 1 wt. % to 30 wt. % of a disintegrant, about 1 wt. % to 30 wt. % of a surfactant, and about 1 wt. % to 30 wt. % of a lubricant.

In one embodiment, the formulation comprises about 20 wt. % to 75 wt. % of the co-crystal, about 20 wt. % to 75 wt. % of a filler, about 1 wt. % to 10 wt. % of the polymer, about 1 wt. % to 10 wt. % of a disintegrant, about 1 wt. % to 10 wt. % of a surfactant, and about 1 wt. % to 10 wt. % of a lubricant.

In certain formulations, the formulation comprises one or more excipients. The one or more excipients comprises silicifed microcrystalline cellulose, croscarmellose sodium, sodium lauryl sulfate, HPMC, lactose and sodium stearyl fumarate.

In certain embodiments, the formulation is in a form of a capsule, tablet, pill, powder, granule, aqueous suspension or solution. In one embodiment, the formulation is in a form of a capsule. Alternatively, in one embodiment, the formulation is in a form of a tablet. In another embodiment, the tablet is coated.

In certain embodiments, the co-former is a carboxylic acid. In certain embodiments, the co-former is salicyclic acid, 4-amino salicylic acid, oxalic acid, 4-hyrdoxybenzoic acid, 2,4 hyrdoxybenzoic acid, 2,5-hyrdoxybenzoic acid and vanillic acid. In certain embodiments, the co-former is 4-hyrdoxybenzoic acid.

In certain embodiments, the API is a protease inhibitor.

In certain embodiments, the solubility of the API in water is less than one part by weight of the API in fifty parts by weight of water. In certain embodiments, the solubility of the API in water is less than one part by weight of the API in hundred parts by weight of water.

In certain embodiments, the API is VX-950, as shown below:

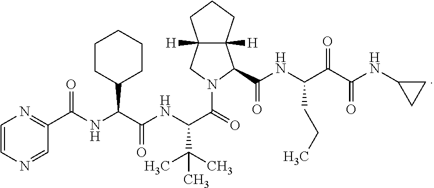 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

In another aspect, the present invention is a method of formulating a co-crystal. The method includes steps of:
1) at least partially dissolving an aqueous component in an aqueous-based solution;
2) contacting the aqueous-based solution with a co-crystal component; and
3) obtaining a substantially dry blend comprising the co-crystal component and the hydrophilic component.
   The kinetic solubility of the co-crystal after being in contact with an environment of use is at a therapeutically acceptable level for a prolonged period of time. The aqueous component includes a surfactant and, optionally, a polymer. The co-crystal component includes a co-crystal, a filler and, optionally, a polymer, provided that one of the co-crystal component and the aqueous component includes the polymer. Furthermore, the co-crystal includes i) a co-former and ii) an API with solubility in water is less than one part by weight of the API in ten parts by weight of water. The formulation comprising particles of the co-crystal and the polymer in intimate association with each other, and the kinetic solubility of the co-crystal after being in contact with an environment of use for one hour is greater than 0.100 mg/ml.

In certain embodiments, the method further includes a step of compressing the dry blend into a tablet.

In certain embodiments, the method further includes processing the substantially dry blend.

In certain embodiments, the step of contacting the aqueous-based solution with a co-crystal component includes granulating the aqueous component and the co-crystal component. In certain embodiments, the step of granulating is fluid bed granulation.

In another aspect of the present invention includes an aqueous-based granulation. The aqueous-based granulation includes:

a co-crystal, the co-crystal comprising:
a) a co-former; and
b) an active pharmaceutical ingredient (API)

a polymer.

The co-crystal comprises at least 5% of the total weight of a substantially dry mass of the aqueous-based granulation. Furthermore, at least 1% of the total weight of the aqueous-based granulation is water, and the co-former and the API of the co-crystal do not dissociate when come in contact with water.

In certain embodiments, the co-crystal comprises at least 50% of the total weight of a substantially dry mass of the aqueous-based granulation. In certain embodiments, the co-crystal comprises at least 75% of the total weight of a substantially dry mass of the aqueous-based granulation. In certain embodiments, the co-crystal comprises at least 90% of the total weight of a substantially dry mass of the aqueous-based granulation. In certain embodiments, the co-crystal comprises at least 99% of the total weight of a substantially dry mass of the aqueous-based granulation.

In certain embodiments, at least 10% of the total weight of the aqueous-based granulation is water. In certain embodiments, at least 25% of the total weight of the aqueous-based granulation is water. In certain embodiments, at least 45% of the total weight of the aqueous-based granulation is water.

In certain embodiments, the solubility of the API in water is less than one part by weight of the API in ten parts by weight of water.

In certain embodiments, the aqueous-based granulation further comprises one or more excipients.

In certain embodiments, the one or more excipients is added to the aqueous-based granulation, and the one or more excipients includes one or more selected from the group consisting of: a filler, a surfactant, a diluent, a binder and a disintegrant.

In certain embodiments, the one or more excipients includes one or more fillers.

In certain embodiments, the aqueous-based granulation comprises about up to 25 wt. % of the one or more fillers. In other embodiments, the aqueous-based granulation comprises about up to 50 wt. % of the one or more fillers. Yet in other embodiments, the aqueous-based granulation comprises about up to 10 wt. % of the one or more fillers.

In certain embodiments, the one or more fillers of the aqueous-based granulation is selected from one or more from the group consisting of the following: mannitol, lactose, sucrose, dextrose, maltodextrin, sorbitol, xylitol, powdered cellulose, microcrystalline cellulose, silicified microcrystalline cellulose, methylcellulose, ethylcellulose, hydroxyethylcellulose, methylhydroxyethylcellulose, talc, starch, pregelatinized starch, dibasic calcium phosphate, calcium sulfate and calcium carbonate. In certain embodiments, the one or more fillers is a cellulosic filler. In certain embodiments, the one or more fillers is microcrystalline cellulose or lacotse. Yet on other embodiments, the one or more fillers is microcrystalline cellulose and lactose.

In certain embodiments, the aqueous-based granulation comprises about up to 10 wt. % of the one or more disintegrants. In other embodiments, the aqueous-based granulation comprises about up to 2 wt. % of the one or more disintegrants. In certain embodiments, the one or more excipients of the aqueous-based granulation includes one or more disintegrants. In certain embodiments, the one or more disintegrants is selected from one or more from the group consisting of the following: croscarmellose sodium, sodium alginate, calcium alginate, alginic acid, starch, pregelatinized starch, sodium starch glycolate, crospovidone, cellulose and its derivatives, carboxymethylcellulose calcium, carboxymethylcellulose sodium, soy polysaccharide, guar gum, an ion exchange resin, an effervescent system based on food acids and an alkaline carbonate component, and sodium bicarbonate. In certain embodiments, the one or more disintegrants is croscarmellose sodium. In certain embodiments, the aqueous-based granulation

In certain embodiments, the one or more excipients of the aqueous-based granulation includes one or more diluents. In certain embodiments, the aqueous-based granulation comprises up to about 10 wt. % of the one or more diluents. In certain embodiments of the aqueous-based granulation, the one or more diluents is lactose monohydrate.

In certain embodiments of the aqueous-based granulation, the one or more excipients includes one or more surfactants. In one embodiment, the aqueous-based granulation comprises up to about 10 wt. % of the one or more surfactants. In another embodiment, the aqueous-based granulation comprises up to about 2 wt. % of the one or more surfactants. Yet in another embodiment, the one or more surfactants is selected from one or more from the group consisting of the following: sodium lauryl sulfate, docusate sodium, polyoxyethylene sorbitan fatty acid esters, polyoxyethylene 20 stearyl ethers, polyoxyethylene alkyl ethers, polyoxyethylene castor oil derivatives, pegylated hydrogenated castor oils, sorbitan esters of fatty acids, Vitamin E or tocol derivatives, vitamin E TPGS, tocopheryl esters, lecithin, phospholipids and their derivatives, poloxamers, stearic acid, oleic acid, oleic alcohol, cetyl alcohol, mono and diglycerides, propylene glycol esters of fatty acids, glycerol esters of fatty acids, ethylene glycol palmitostearate, polyoxylglycerides, propylene glycol monocaprylate, propylene glycol monolaurate and polyglyceryl oleate. In another embodiment, the one or more surfactants is sodium lauryl sulfate.

In certain embodiments of the aqueous-based granulation, the polymer is selected from the following: hydroxypropylmethyl cellulose (HPMC), hydroxypropylmethyl cellulose acetate succinate (HPMCAS), polyvinylpyrrolidone (PVP), polyethylene glycol (PEG), poly(ethylene oxide) (PEO), Polyvinylpyrrolidone-co-vinylacetate (PVPVA), poly(meth)acrylate (e.g., Eudragit® polymers), Polyvinylpyrrolidone (PVP), Polyvinylacetate (PVA) or a combination thereof. In one embodiment, the polymer is HPMC. In another embodiment, the polymer comprises 0.1 wt. % to 10 wt. % of the total weight of the granulation.

In certain embodiments, the aqueous-based granulation further comprises sodium lauryl sulfate. In one embodiment, the aqueous-based granulation further comprises one of more of the following: microcrystalline cellulose, croscarmellose sodium, lactose monohydrate, and sodium stearyl fumarate.

In certain embodiments of the aqueous-based granulations, the co-former of the co-crystal is a carboxylic acid. In one embodiment, the co-former is 4-hyrdoxybenzoic acid.

In certain embodiments of the aqueous-based granulations, the API of the co-crystal is a protease inhibitor.

In certain embodiments, the solubility of the API in water is less than one part by weight of the API in fifty parts by weight of water. In certain embodiments, the solubility of the API in water is less than one part by weight of the API in 100 parts by weight of water. In certain embodiments, the API is VX-950.

In certain embodiments, the aqueous-based granulation is shear granulated. In certain embodiments, the aqueous-based granulation is high shear granulated.

In certain embodiments, the aqueous-based granulation is fluid bed granulated.

Another aspect the present invention is a drug dosage form comprising the aqueous-based granulation described herein.

In certain embodiments, the drug dosage form is substantially free of water. In certain embodiments, the dosage form is in a form of a capsule, tablet, pill, powder, and granule. In one embodiment, the dosage form is in a form of a capsule. In another embodiment, the dosage form is in a form of a tablet. In yet another embodiment, the tablet is coated. In another embodiment, the API comprises at least 50% of the total weight of the dosage form.

Another aspect of the present invention is a method of formulating a drug dosage form of a co-crystal. The method includes the steps of
1) at least partially dissolving the co-crystal in an aqueous-based solution, wherein the aqueous component includes a polymer;
2) contacting the aqueous-based solution with a co-crystal component, wherein the co-crystal component includes:
   a) a co-crystal, wherein the co-crystal comprising
      i) a co-former; and
      ii) an API.
         The co-crystal comprises at least 5% of the total weight of a substantially dry mass of the aqueous based granulation. At least 1% of the total weight of the aqueous-based granulation is water, and the co-former and the API of the co-crystal do not dissociate when come in contact with water.

In one embodiment, the solubility of the API in water is less than one part by weight of the API in ten parts by weight of water. In another embodiment, the solubility of the API in water is less than one part by weight of the API in fifty parts by weight of water. Yet in another embodiment, the solubility of the API in water is less than one part by weight of the API in 100 parts by weight of water.

In certain embodiments, the API is VX-950.

In certain embodiments, the granulation is shear granulated. In one embodiment, the granulation is high shear granulated. In another embodiment, the granulation is fluid bed granulated.

### DETAILED DESCRIPTION OF THE INVENTION

It must be noted that as used herein and in the claims, the singular forms “a,” “an,” and “the” include plural referents unless the context clearly dictates otherwise. Thus, for example, reference to “a binder” includes two or more binders; reference to “a pharmaceutical agent” includes two or more pharmaceutical agents, and so forth.

As used herein, the term “active agent,” “active pharmaceutical ingredient” or “API” refers to a pharmaceutically active agent or a drug, and all these terms may be used interchangeably. Furthermore, these terms can also refer to a co-crystal that includes VX-950 specifically.

The term “co-crystal” as used herein means one unique solid form of a crystalline material comprised of two or more unique compounds, the two or more unique compounds forming a new chemical entity. The co-crystal is a solid at room temperature, and has physically and chemically distinct characteristics from each of the two or more unique compounds. As mentioned above, co-crystals of this invention can be analyzed by methods known in the art for characterizing solid or crystalline materials. Examples of characterization methods include thermogravimetric analysis (TGA), differential scanning calorimetry (DSC), X-ray powder diffraction (XRPD), solubility analyses, dynamic vapor sorption, infrared off-gas analysis, and suspension stability. TGA can be used to investigate the presence of residual solvents in a co-crystal sample, and to identify the temperature at which decomposition of each co-crystal sample occurs. DSC can be used to look for thermo-transitions occurring in a co-crystal sample as a function of temperature and determine the melting point of each co-crystal sample. XRPD can be used for structural characterization of the co-crystal.

The co-crystals of the present invention comprise a co-former non-covalently bound to an API (i.e., VX-950). For example, the co-former may be H-bonded directly to the API or may be H-bonded to an additional molecule which is bound to the API. The additional molecule interacts with the API non-covalently. The additional molecule can also be a different API. Solvates of API compounds that do not further comprise a co-former are not co-crystals according to the present invention. The co-crystals may however, include one or more solvent molecules in the crystalline lattice. That is, solvates of co-crystals, or a co-crystal further comprising a solvent or compound that is a liquid at room temperature, is included in the present invention, but crystalline material comprised of only one solid and one or more liquids (at room temperature) are not included in the present invention, with the previously noted exception of specifically stated liquid APIs. The co-crystals may also be a co-crystal between a co-former and a salt of an API, but the API and the co-former of the present invention are constructed or bonded together through hydrogen bonds. Other modes of molecular recognition may also be present including, pi-stacking, guest-host complexation and van der Waals interactions. Of the interactions listed above, hydrogen-bonding is the dominant interaction in the formation of the co-crystal, (and a required interaction according to the present invention) whereby a non-covalent bond is formed between a hydrogen bond donor of one of the moieties and a hydrogen bond acceptor of the other. Hydrogen bonding can result in several different intermolecular configurations. For example, hydrogen bonds can result in the formation of dimers, linear chains, or cyclic structures. These configurations can further include extended (two-dimensional) hydrogen bond networks and isolated triads.

The term “co-former” is a pharmacologically inert molecule that alters the crystal form of a solid drug through the formation of co-crystals, clathrates or other crystalline solid forms.

As used herein, an “environment of use” can be either the in vivo environment, such as the GI tract of an animal, particularly a human, or the in vitro environment of a test solution, such as appropriate dissolution media (e.g., phosphate buffered saline solution, 1% sodium lauryl sulfate, fed or fasted simulated gastric or intestinal media). Examples of simulated media include Fed simulated intestinal fluid (FESSIF), Fasted simulated intestinal fluid (FASSIF), Fed simulated gastric Fluid (FESSGF) and Fasted simulated gastric fluid (FASSGF).

The term “excipient” herein includes any substance used as a vehicle for delivery of the active ingredient to a subject, and any substance added to the active ingredient, for example to improve its handling properties or to permit the resulting composition to be formed into an orally deliverable unit dose having the desired shape and consistency. Excipients can include, by way of illustration and not by limitation, a filler, a binder, a surfactant, a disintegrant, a glidant, a lubricant or a combination thereof, dyes, substances added to improve appearance of a dosage form, and any other substance other than the active ingredient conventionally used in the preparation of oral dosage forms.

The term “bioavailability” herein relates to a measure of the amount of active ingredient that is absorbed via the gastrointestinal tract into the bloodstream. More specifically, “bioavailability” is used herein to denote dose-normalized AUC_{(0-∞)} for a specific orally administered composition expressed as a percentage of dose-normalized AUC_{(0-∞)} for the active ingredient delivered intravenously at the same dosage rate. The make-up of the formulation can influence bioavailability of the active ingredient. While two formulations may use the identical active ingredient, the bioavailability of the active ingredient may not be the same. As such, bioavailability of an active ingredient can vary significantly depending on the make-up of a formulation, for example, the ingredients of excipients and their amounts and grade.

In certain embodiment, the composition of the present invention is designed to release of an active ingredient containing a co-crystal that includes a poorly soluble API upon exposure to an environment of use, such as gastric fluid, upon administration. The formulation of the present invention provides for both dissolution of the API upon introduction of the composition to the environment of use, and for a rise in plasma concentration of the practically insoluble active ingredient to therapeutic levels following administration to a subject. Furthermore, once the API is dissolved in the environment, the formulation of the present invention keeps the API supersaturated for a prolonged period of time.

The formulations of the present invention maintain a kinetic solubility at a certain level in a prolonged period of time (e.g., 1.5 hr, 2 hr, 2.5, hr, 3 hr, 3.5 hr, 4 hr, or 4.5 hr, etc.). The certain level of the kinetic solubility can include a level that is therapeutically effective.

It has been found that the co-crystal (e.g., VX-950) and a co-former (e.g., 4-hydroxybenzoic acid) give rise to improved properties, as compared to the crystalline form of the API (including free acids, free bases, and zwitter ions, hydrates, solvates, etc.), particularly with respect to, but are not limited to: solubility, dissolution, bioavailability, stability, Cₘₐₓ, Tₘₐₓ, and processability. For example, a co-crystal that includes VX-950 is advantageous where the free form of crystalline VX-950 is very slightly soluble in water. Additionally, the co-crystal properties conferred upon the API are also useful because the bioavailability of the API can be improved and the plasma concentration and/or serum concentration of the API can be improved. This is particularly advantageous for orally-administrable formulations.

For example, in preparing the co-crystal that includes VX-950 and the co-former of 4-hydroxybenzoic acid, VX-950 and 4-hydroxybenzoic acid are dissolved in an appropriate solvent system. In one embodiment, VX-950 and 4-hydroxy benzoic acid are dissolved independently in respective solvents and subsequently combined. In another embodiment, VX-950 and 4-hydroxy benzoic acid are dissolved together in a mixture of two or more solvents. Seeding can be facilitated formation of the co-crystal, providing better control (e.g., better morphology).

Example of the solvent systems for preparing the co-crystal including VX-950 may include, but are not limited to, ether solvents (e.g., diethyl ether, methyl tert-butyl ether, 2-methyltertahydrofuran, and tetrahydrofuran), acetate solvents (e.g., methyl acetate, ethyl acetate, isopropyl acetate, isobutyl acetate, and tert-butyl acetate), ketone solvents (e.g., acetone, 2-butanone, and methyl isobutyl ketone), alkylhalide solvents (e.g., dichloromethane, chloroform, and dichloroethane), nitrile solvents (e.g., acetonitrile and butyronitrile), or hydrocarbon solvents (e.g., toluene and benzene).

Examples of characterization methods include thermogravimetric analysis (TGA), differential scanning calorimetry (DSC), X-ray powder diffraction (XRPD), single crystal structure determination, solubility analyses, dynamic vapor sorption, infrared off-gas analysis, and suspension stability. TGA can be used to investigate the presence of residual solvents in a co-crystal sample, and to identify the temperature at which decomposition of each co-crystal sample occurs. DSC can be used to look for thermo-transitions occurring in a co-crystal sample as a function of temperature and determine the melting point of each co-crystal sample. XRPD and single crystal structure determination can be used for structural characterization of the co-crystal. Solubility analysis can be performed to reflect the changes in the physical state of each co-crystal sample. And suspension stability analysis can be used to determine the chemical stability of a co-crystal sample in a solvent.

In some embodiment, an effective amount is the amount which is required to confer a therapeutic effect on the treated subject, e.g., a patient. The effective amount of a co-crystal that includes VX-950 and the co-former is between about 0.1 mg/kg to about 150 mg/kg (e.g., from about 1 mg/kg to about 60 mg/kg). Effective doses will also vary, as recognized by those skilled in the art, dependent on route of administration, excipient usage, and the possibility of co-usage with other therapeutic treatments including use of other therapeutic agents and/or therapy.

The co-crystals or pharmaceutical compositions of this invention can be administered to the subject in need thereof (e.g., cells, a tissue, or a patient (including an animal or a human)) by any method that permits the delivery of the compound VX-950, e.g., orally, intravenously, or parenterally. For instance, they can be administered via pills, tablets, capsules, aerosols, suppositories, liquid formulations for ingestion or injection or for use as eye or ear drops, dietary supplements, and topical preparations.

In some embodiments, the present invention provides methods of controlling the particle size of the co-crystals. The particle size of the co-crystal disclosed herein can influence physical and chemical features (e.g., bioavailability) of the formulations of the present invention. Depending on the type of machines (e.g., Jetmill® MC50 Jetpharma Micronizer) used to micronize the co-crystal, the particle size of the co-crystal can vary. Examples of controlling the particle sizes of the co-crystal can include, but are not limited to, the following: crushing the co-crystal, sieving the co-crystal, and milling the co-crystal. Examples of milling include, but not limited to, fitzmilling, co-milling, jet-milling, wet-milling, nano-milling, or a combination thereof.

In one embodiment, the pharmaceutical formulations of the present invention include one or more excipients. In certain embodiment, the one more excipients include one or more fillers.

The term “filler component” refers to one or more substances that act to dilute the API to the desired dosage and/or that act as a carrier for the API. In some embodiments of the pharmaceutical formulations, the first filler component comprises one or more filler substances. In some embodiments of the pharmaceutical formulations, the filler component comprises one or more diluent substances. In some embodiments of the pharmaceutical formulations, the first filler component comprises one or more substances that are diluents and fillers. In some embodiments, the first filler component comprises at least one a substance that improves the mechanical strength and/or compressibility of the pharmaceutical compositions of the invention.

Examples of the filler components can include, but are not limited to, mannitol, lactose (e.g., Lactose—316 Fast-Flo), sucrose, dextrose, maltodextrin, sorbitol, xylitol, powdered cellulose, microcrystalline cellulose (e.g., Avicel PH113, PH101, PH102, etc.), silicified microcrystalline cellulose (e.g., Prosolv HD90), methylcellulose, ethylcellulose, hydroxyethylcellulose, methylhydroxyethylcellulose, talc, starch, pregelatinized starch, dibasic calcium phosphate, calcium sulfate and calcium carbonate.

In some embodiments of the pharmaceutical formulations, the filler is microcrystalline cellulose, silicified microcrystalline cellulose or lactose.

In some embodiments of the pharmaceutical formulations, the filler is silicified microcrystalline cellulose.

In some embodiments, the filler is present in an amount of about least 1, 2, 3, 4, 5, 6, 7, 8, 9, 10, 11, 12, 13, 14, 15, 20, 25, 30, 35, 40, 45, 50, 55 or 60% of the total weight of the formulation. In some embodiments of the pharmaceutical formulations, the filler component comprises from about 20% to about 55% by weight of the pharmaceutical formulation. In some embodiments of the pharmaceutical formulations, the filler component comprises from 35% to about 50% by weight of the pharmaceutical formulation. In some embodiments of the pharmaceutical formulations, the filler component comprises from 45% to about 50% by weight of the pharmaceutical formulation.

Examples of the disintegrants may include, but are not limited to, croscarmellose sodium (e.g., AcDiSol), sodium alginate, calcium alginate, alginic acid, starch, pregelatinized starch, sodium starch glycolate, crospovidone, carboxymethylcellulose calcium, cellulose and its derivatives, carboxymethylcellulose sodium, soy polysaccharide, guar gum, an ion exchange resin, an effervescent system based on food acids and an alkaline carbonate component, and sodium bicarbonate.

In some embodiments of the pharmaceutical formulations, the disintegrant component is croscarmellose sodium.

In some embodiments of the pharmaceutical formulations, the disintegrant component comprises an amount of about least 1, 2, 3, 4, 5, 6, 7, 8, 9, 10, 15, 20, 25, 30, or 40% of the total weight of the formulation. In some embodiments of the pharmaceutical formulations, the disintegrant component comprises from about 1% to about 20% by weight of the pharmaceutical formulation. In some embodiments, the disintegrant component comprises from about 1% to about 15% by weight of the pharmaceutical formulation. In some embodiments, the disintegrant component comprises from about 1% to about 10% by weight of the pharmaceutical formulation. In some embodiments, the disintegrant component comprises from about 1% to about 5% by weight of the pharmaceutical formulation. In some embodiments, the disintegrant component comprises from about 2% to about 5% by weight of the pharmaceutical formulation.

In certain embodiments of the pharmaceutical formulations, the one more excipients include one or more surfactants. Surfactants may be used to enhance wettability of poorly soluble or hydrophobic compositions. The surfactants can include, but are not limited to, ionic surfactants, non-ionic surfactants or cationic surfactants. Examples of the surfactants may include, but are not limited to, sodium lauryl sulfate, docusate sodium, polyoxyethylene sorbitan fatty acid esters (e.g., polysorbate/Tween 20, 40, 60 and 80), polyoxyethylene 20 stearyl ethers (also known as the Brij series of surfactants; e.g., Brij 78, Brij 30, Brij 35, Brij 52, Brij 56, Brij 58, Brij 72, Brij 721, Brij 76, Brij 92, Brij 96, and Brij 98), polyoxyethylene alkyl ethers, polyoxyethylene castor oil derivatives (e.g., polyoxyl 35 castor oil e.g., CREMOPHOR® EL, polyoxyl 40 hydrogenated castor oil e.g., CREMOPHOR® RH40), pegylated hydrogenated castor oils, sorbitan esters of fatty acids (e.g., sorbitan monolaurate e.g., Span 20, sorbitan monooleate e.g., Span 80, sorbitan monopalmitate e.g., Span 40, sorbitan monostearate e.g., Span 60, sorbitan tristearate etc.), Vitamin E or tocol derivatives, vitamin E TPGS, tocopheryl esters, natural or synthesized lecithins, phospholipids and their derivatives, poloxamers(polyoxyethylene and polyoxypropylene copolymers, e.g., poloxamer 407, poloxamer 388, poloxamer 188), stearic acid, oleic acid, oleic alcohol, cetyl alcohol, mono and diglycerides, propylene glycol esters of fatty acids, glycerol esters of fatty acids (e.g., glycerol monooleate, glycerol monostearate), ethylene glycol palmitostearate, polyoxylglycerides (pegylated glycerides, e.g., labrafils, labrasol, gelucires etc.), propylene glycol monocaprylate, propylene glycol monolaurate, polyglyceryl oleate, or any combination of the herein above-mentioned surfactants.

In certain embodiment of the pharmaceutical formulations, the one or more surfactant is sodium lauryl sulfate.

In some embodiments of the pharmaceutical formulations, the one or more surfactant comprises an amount of about least 1, 2, 3, 4, 5, 6, 7, 8, 9, 10, 15, 20, 25, 30, or 40% of the total weight of the formulation. In some embodiments of the pharmaceutical formulations, the one or more surfactants component comprises from about 1% to about 20% by weight of the pharmaceutical formulation. In some embodiments, the one or more surfactants component comprises from about 1% to about 15% by weight of the pharmaceutical formulation. In some embodiments, the one or more surfactants component comprises from about 1% to about 10% by weight of the pharmaceutical formulation. In some embodiments, the one or more surfactants component comprises from about 1% to about 5% by weight of the pharmaceutical formulation. In some embodiments, the one or more surfactants component comprises from 2% to about 5% by weight of the pharmaceutical formulation.

In certain embodiments, the one more excipients can include one or more lubricants. Suitable lubricants possess anti-sticking or anti-tacking properties. Examples of the lubricants may include, but are not limited to, talc, fatty acid, stearic acid, magnesium stearate, calcium stearate, sodium stearate, glyceryl monostearate, sodium lauryl sulfate, sodium stearyl fumarate, hydrogenated oils, fatty alcohol, fatty acid ester, glyceryl behenate, mineral oil, vegetable oil, leucine, sodium benzoate, or a combination thereof. In certain embodiment of the pharmaceutical formulations, the one or more lubricant is sodium stearyl fumarate.

In some embodiments of the pharmaceutical formulations, the one or more lubricant comprises an amount of about least 1, 2, 3, 4, 5, 6, 7, 8, 9, 10, 15, 20, 25, 30, 35 or 40% of the total weight of the formulation. In some embodiment, the one or more lubricant comprises from about 1% to about 20% by weight of the pharmaceutical formulation. In some embodiments, the one or more lubricant comprises from about 1% to about 15% by weight of the pharmaceutical formulation. In some embodiments, the one or more lubricant comprises from about 1% to about 10% by weight of the pharmaceutical formulation. In some embodiments, the one or more lubricant comprises from about 1% to about 5% by weight of the pharmaceutical formulation. In some embodiments, the one or more lubricant comprises from about 2% to about 5% by weight of the pharmaceutical formulation.

In certain embodiments, the one more excipients can include one or more glidants. Suitable glidants can improve the flow of a formulation, and may possess anti-sticking or anti-tacking properties. Examples of the glidants may include, but are not limited to, talc, colloidal silica (e.g., Cabosil M-5), magnesium oxide, magnesium silicate, leucine and starch or a combination thereof. In certain embodiment of the pharmaceutical formulations, the one or more glidant is colloidal silica.

In some embodiments of the pharmaceutical formulations, the one or more glidant component comprises from about 1% to about 30% by weight of the pharmaceutical formulation.

In some embodiments, the one or more glidant component comprises from about 1% to about 20% by weight of the pharmaceutical formulation. In some embodiments, the one or more glidant component comprises from 1% to about 10% by weight of the pharmaceutical formulation. In some embodiments, the one or more glidant component comprises from about 1% to about 5% by weight of the pharmaceutical formulation. In some embodiments, the one or more glidant component comprises from about 1% to about 3% by weight of the pharmaceutical formulation. In some embodiments, the one or more glidant component comprises from about 2% to about 3% by weight of the pharmaceutical formulation. In some embodiments, the one or more glidant component comprises about 3% by weight of the pharmaceutical formulation. In some embodiments, the one or more glidant component comprises about 3% by weight of the pharmaceutical formulation. In some embodiments, the one or more glidant component comprises about 3% by weight of the pharmaceutical formulation.

In certain embodiments, the polymer is selected from the following: hydroxypropylmethyl cellulose (HPMC), hydroxypropylmethyl cellulose acetate succinate (HPMCAS), polyvinylpyrrolidone (PVP), polyethylene glycol (PEG), poly(ethylene oxide) (PEO), Polyvinylpyrrolidone-co-vinylacetate (PVPVA), poly(meth)acrylate (e.g., Eudragit® polymers), Polyvinylpyrrolidone (PVP), Polyvinylacetate (PVA) or a combination thereof. In certain embodiment, the polymer is HPMCAS or HPMC. In certain embodiment, the polymer is HPMC. In certain embodiments, the polymer comprises 0.1 wt. % to 30 wt. % of the formulation. In certain embodiments, the polymer comprises 1 wt. % to 20 wt. % of the formulation. In certain embodiments, the polymer comprises 1 wt. % to 15 wt. % of the formulation. In certain embodiments, the polymer comprises 1 wt. % to 10 wt. % of the formulation. In certain embodiments, the polymer comprises 1 wt. % to 7 wt. % of the formulation. In certain embodiments, the polymer comprises 1 wt. % to 5 wt. % of the formulation. In certain embodiments, the polymer is a different grade HPMC. The different grades of HPMC include E50, E15, E3, E5, and others. In one embodiment, the polymer is HPMC E50 (See METHOCEL Cellulose Ethers—Technical Handbook by Dow Chemical, September, 2002; herein incorporated by reference.)

In certain embodiments, the formulations of the present invention comprise one or more excipients selected from the group consisting of: the co-crystal, silicified microcrystalline cellulose (e.g., Prosolv 90), HPMC, croscarmellose sodium (e.g., Ac-Di-Sol), sodium lauryl sulfate, sodium stearyl fumarate or a combination thereof.

The pharmaceutical compositions of this invention may be orally administered in any orally acceptable dosage form including, but are not limited to, capsules, tablets, pills, powders, granules, aqueous suspensions or solutions. Other pharmaceutical compositions of the present invention (as well as compositions for use in methods, combinations, kits, and packs of the present invention) may be administered orally, parenterally, sublingually, by inhalation spray, topically, rectally, nasally, buccally, vaginally or via an implanted reservoir. The term “parenteral” as used herein includes subcutaneous, intravenous, intramuscular, intra articular, intra synovial, intrasternal, intrathecal, intrahepatic, intralesional and intracranial injection or infusion techniques. Preferably, the compositions are administered orally or intravenously.

In certain embodiments, the kinetic solubility of the formulation described herein is at least 0.15 mg/ml for a prolonged period of time (e.g., 1.5 hr, 2 hr, 2.5, hr, 3 hr, 3.5 hr, 4 hr or 4.5 hr, etc.). In certain embodiments, the kinetic solubility of the formulation described herein is at least 0.20 mg/ml, 0.30 mg/ml, 0.40 mg/ml, 0.50 mg/ml or 0.60 mg/ml for a prolonged period of time.

Such administration can be used as a chronic or acute therapy. The amount of active ingredient that may be combined with the carrier materials to produce a single dosage form will vary depending upon the host treated and the particular mode of administration. The particular dose unit can be selected to accommodate the desired frequency of administration used to achieve a desired daily dosage. The daily dosage and frequency of administration, and therefore the selection of appropriate dose unit, depends on a variety of factors, including the age, weight, sex and medical condition of the subject, and the nature and severity of the condition or disorder, and thus may vary widely.

The API and excipient(s) mixture can be prepared by, for instance, conventional mixing, compacting, granulating, compression, or coating. Procedures which may be used are known in the art, e.g., those described in L. Lachman et al. The Theory and Practice of Industrial Pharmacy, 3rd Ed, 1986, H. Sucker et al, Pharmazeutische Technologie, Thieme, 1991, Hagers Handbuch der pharmazeutischen Praxis, 4th Ed. (Springer Verlag, 1971) and Remington's Pharmaceutical Sciences, 13th Ed. (Mack Publ., Co., 1970) or later editions. Examples of such techniques are as follows:

(1) Blending of the co-crystal with the appropriate excipients using different blending equipment, such as low shear blenders and high shear blenders;

(2) Direct compression of the blends, using appropriate punches and dies; the punches and dies are fitted to a suitable compaction machine, such as rotary tableting press or a single station compaction machine;

(3) Some of the formulation ingredients can be granulated if necessary, using appropriate granulation methods such as dry granulation (slugging or roller compaction), high shear wet granulation, fluid bed granulation, extrusion-spheronization, melt extrusion, spray drying;

(4) Granulation followed by compression; and

(5) Coating of the tablets produced using appropriate coating equipment (e.g., coating pans) and appropriate coating solutions/suspensions to be applied on the tablets.

In some embodiments, the present invention is directed a formulation which comprises a co-crystal of a poorly soluble API as described herein in intimate association with a polymer. This can be accomplished, for example, by 1) preparing an aqueous solution or slurry of excipients, which optionally includes a polymer, 2) mixing the slurry with a co-crystal blend, and 3) drying the mixture. This processed product includes the co-crystal and the polymer in intimate association with each other. The relationship between the co-crystal and the polymer can be further in intimate association with other fillers described herein. The processed products are not necessarily uniform or homogeneous.

By “intimate associate” or “intimate association”, it is meant that the polymer has in some manner been integrated with the co-crystal particles, e.g., via a partial coating of the co-crystal particles, as opposed to a chemical interaction of the two ingredients. The term “intimate association” is therefore deemed for purposes of the present description as being synonymous with “integrated” or “united”.

In some embodiments, the formulations of the present invention are processed with mixing a co-crystal component and an aqueous component. The co-crystal component may include a co-crystal of an API and a co-former, a filler and, optionally, a polymer. The aqueous component may include a surfactant and, optionally, a polymer. Here, the aqueous component is at least partially dissolved in an aqueous-based solvent. The term “aqueous-based solvent” herein refers to water or to a cosolvent system that includes water. When the mixing processes of the embodiments involve water, the API and the co-former of the co-crystal, which usually dissociate when contacted water, remain intact despite the presence of water when processed in accordance with the methods disclosed herein. Depending on the characteristics of the desired formulation, the process variables are adjusted to account, for changes in the product particle size, bulk density, and flow characteristics.

The term “granulation” refers to a process of size-enlargement, where primary powder particles are gathered into larger, semi-permanent aggregates (granules). Pharmaceutical granules typically have a size range of 10 μm to 2 mm and the majority will be used as an intermediate product in the production of tablets, while some granulations will be dispensed as such in packets or in capsules. The main granulation methods are dry and wet granulation. Via dry granulation powders are compacted under high pressure using (a) slugging to produce a large tablet (“slug”) in a tabletting machine or (b) roller compaction to compact the powder between two rollers into a material sheet. After both processes the compacted material is broken up by milling to produce the granular material. This dry method can be used for drugs sensitive to moisture. Wet granulation occurs when powders are mixed with a liquid phase during the granulation process. Example of granulations include fluid bed granulation and high-shear granulation.

In certain embodiments, the methods of the present invention are processed using fluid bed granulation. In certain embodiments, the fluid bed granulation is an aqueous-based process.

Formulating a co-crystal drug substance is a challenging endeavor. A co-crystal is formed when a chemical compound hydrogen-bonds and other interactions with a co-former compound. For an example, VX-950 and the co-crystal former (i.e., 4-hydroxybenzoic acid) are held together in the crystalline form. It has now unexpectedly been found that a co-crystal can be stabilized by one or more excipients by, (1) inhibiting dissociation of the co-crystal despite being in contact with water, and/or (2) substantially preventing a practically insoluble API from crystallizing and thereby rendering the API to be dissolved in an environment of use to be bioavailable. As indicated above, when the mixing processes of the embodiments involve water, the co-crystal, which usually dissociates when contacting water, remain intact despite the presence of water when processed in accordance with the methods disclosed herein.

In another embodiment, a wet granulation process is performed to yield the pharmaceutical formulation of the invention from an admixture of powdered and liquid ingredients. For example, a pharmaceutical composition comprising an admixture of a composition comprising the co-crystal of VX-950 and one or more excipients selected from: a filler, a diluent, a binder, a glidant, a surfactant, a lubricant, a disintegrant, are weighed as per the formula set herein. Next, all of the intragranular ingredients are sifted and mixed in a high shear or low shear granulator using water or water with a surfactant or water with a binder or water with a surfactant and a binder to granulate the powder blend. A fluid other than water can also be used with or without surfactant and/or binder to granulate the powder blend. Next, the wet granules can optionally be milled using a suitable mill. Next, water may optionally be removed from the admixture by drying the ingredients in any suitable manner. Next, the dried granules can optionally be milled to the required size. Next, extra granular excipients can be added by blending (for example a filler, a diluent, and a disintegrant). Next, the sized granules can be further lubricated with a lubricant (e.g., magnesium stearate). Next, the lubricated granulation is compressed into a suitable rug dosage form. Optionally, the tablets can be coated with a film, colorant or other coating.

Further, in particular, wet granulation processes (i.e., high shear granulation) disclosed herein, enhancing processibility of a formulation mixture, can allow a formulation to be processed with lower amounts of expicients (i.e., less filler and diluent) rendering more room for the drug substance be included in a drug product and to improve the processibility (e.g., higher density better flow). Furthermore, an increased drug load in a drug dosage form has the additional benefit of providing a smaller tablet size thereby improving tablet pill burden, especially for patients in a combination therapy. Therefore, a drug dosage form of the present invention can provide for the administration of a co-crystal of a low solubility or practically insoluble API in a smaller size than was hitherto possible for a given unit dose of the co-crystal, including that of VX-950. This can lead to a better patient compliance.

In any of these embodiments, the specified amount of the API is administered once a day. Alternatively, the amount of the API is administered twice a day (e.g., BID; q12h). Alternatively, the amount of the API is administered three times a day (e.g., TID; q8h). Further, the API may be administered with or without food. In some embodiments, the API is VX_{—}950.

In certain embodiments, the methods of the present invention are processed using high shear wet granulation. In certain embodiments, the high shear wet granulation is an aqueous-based process.

As indicated above, the formulations of the present invention find their greatest utility when administered to a subject who is in the fed or fasted state, preferably in the fed state.

The tablets may be produced by way of a conventional method or combinations of conventional methods such as roller compaction and direct compression method. For example, a tableting process is essential for production methods of tablets, and also the other processes such as of mixing, drying, and coating may be combined as required.

In one embodiment of the invention, the tablet has a hardness in the range of about 4 to 20 kp (kilopond). The tablet of this embodiment may or may not comprise an outer coating as described below. In another embodiment, the tablet preferably has a hardness in the range of about 10 to 20 kp.

Yet in one embodiment of the present invention, the formulation includes tablet compositions that may be coated.

Specific embodiments of the present invention are illustrated by way of the following examples. This invention is not confined to the specific limitations set forth in these examples, but rather to the scope of the appended claims. Unless otherwise stated, the percentages and ratios given below are by weight.

### EXAMPLES

### Example 1

### Procedure for Making a Tablet

A quantity of VX-950 cocrystal and Prosolv (silicified microcrystalline cellulose) is blended in the mixer. The blend is placed into fluid bed granulator chamber. An aqueous solution containing HPMC E50 and sodium lauryl sulfate (SLS) was made and pumped to the nozzle of the fluid bed granulator. The co-crystal blend from the chamber is fluidized upward with air and exposed to the aqueous solution from the nozzle. Once the desired weight percentage of HPMC E50 and SLS is applied the granules are dried inside the chamber for a period of time. Dried fluid bed granules are blended with Ac-Di-Sol (croscarmellose sodium), Avicel (microcrystalline cellulose) and lactose until a uniform mixture is formed. Sodium stearyl fumarate (SSF) is added to the blend and blended again for a period of time. The tablets are compressed on the rotary press.

TABLE 1
Composition of the tablet:

| Material | wt % |
|---|---|
| VX-950 co-crystal | 33.6 |
| Prosolv | 33.6 |
| HPMC E50 | 4.5 |
| SLS | 2.2 |
| SSF | 3 |
| AcDiSol | 3 |
| Avicel | 10 |
| Lactose | 10 |

### Example 2

Kinetic solubility profiles of VX-950 co-crystal fluid bed granules were determined in Fasted State Simulated Intestinal Fluid (FASSIF).

166.75 mg of VX-950 co-crystal fluid-bed granules were dissolved in 100 ml of FASSIF. Kinetic solubility of VX-950 co-crystal fluid bed granules was measured after 30 min, 1 hr, 2 hr and 4 hr, as shown in Table 2.

TABLE 2

| Time (min) | VX-950 concentration (mg/ml) in FASSIF |
|---|---|
| 30 | 0.124 |
| 60 | 0.122 |
| 120 | 0.119 |
| 240 | 0.118 |

### Example 3

Kinetic solubility profiles of VX-950 co-crystal fluid bed granules were determined in Fasted State Simulated Gastric Fluid (FASSGF).

166.35 mg of VX-950 co-crystal fluid-bed granules were dissolved in 100 ml of FASSGF. Kinetic solubility of VX-950 co-crystal fluid bed granules was measured after 30 min, 1 hr, 2 hr and 4 hr, as shown in Table 3.

TABLE 3

| Time (min) | VX-950 concentration (mg/ml) in FASSGF |
|---|---|
| 30 | 0.355 |
| 60 | 0.382 |
| 120 | 0.354 |
| 240 | 0.210 |

### Example 4

Kinetic solubility profiles of VX-950 co-crystal fluid bed granules were determined in Fed State Simulated Gastric Fluid (FESSGF).

166.25 mg of VX-950 co-crystal fluid-bed granules were dissolved in 100 ml of FESSGF. Kinetic solubility of VX-950 co-crystal fluid bed granules was measured after 30 min, 1 hr, 2 hr and 4 hr, as shown in Table 4.

TABLE 4

| Time (min) | VX-950 concentration (mg/ml) in FESSGF |
|---|---|
| 30 | 0.232 |
| 60 | 0.309 |
| 120 | 0.392 |
| 240 | 0.468 |

### Example 5

Kinetic solubility profiles of VX-950 co-crystal fluid bed granules were determined in Fed State Simulated Intestinal Fluid (FESSIF).

166 94 mg of VX-950 co-crystal fluid-bed granules were dissolved in 100 ml of FESSIF. Kinetic solubility of VX-950 co-crystal fluid bed granules was measured after 15 min, 30 min, 45 min, 1 hr, 1 hr30 min, and 3 hr, as shown in Table 5.

TABLE 5

| Time (min) | VX-950 concentration (mg/ml) in FESSIF |
|---|---|
| 15 | 0.096 |
| 30 | 0.113 |
| 45 | 0.119 |
| 60 | 0.121 |
| 90 | 0.125 |
| 180 | 0.128 |

### Example 6

The experiment of Example was repeated with 166.51 mg of VX-950 co-crystal fluid-bed granules. Kinetic solubility is shown in Table 6

TABLE 6

| | VX-950 concentration (mg/ml) in |
|---|---|
| Time (min) | FESSIF #1 |
| 15 | 0.106 |
| 30 | 0.118 |
| 45 | 0.122 |
| 60 | 0.125 |
| 90 | 0.128 |
| 180 | 0.129 |

All publications and patents cited in this specification are herein incorporated by reference as if each individual publication or patent were specifically and individually indicated to be incorporated by reference. Although the foregoing invention has been described in some detail by way of illustration and example for purposes of clarity or understanding, it will be readily apparent to those of ordinary skill in the art in light of the teachings of this invention that certain changes and modifications may be made thereto without departing from the spirit or scope of the appended claims.

### Example 7

### In Vivo

The dose normalized AUC of Co-crystal tablets containing fluid bed granules dosed in male beagle dogs at 375 mg of VX-950 was: mean value of 514 heng/ml with a standard deviation of 249.8.

### Example 8

Kinetic solubility of the following substances or formulations were measured in FESSIF:

a) A fluid Bed granulated VX-950 formulation;

b) A blended VX-950 formulation;

c) Crystalline VX-950; and

d) Pure co-crystal of VX-950.

TABLE 7

| | Fluid Bed | | | |
|---|---|---|---|---|
| | Granules with VX- | Crystalline | Blend with | VX-950 |
| Time | 950 cocrystal | VX-950 | cocrystal | cocrystal |
| [min] | [mg/ml] | [mg/ml] | [mg/ml] | [mg/ml] |
| 15 | 0.101 | 0.005 | 0.085 | 0.0925 |
| 30 | 0.116 | 0.005 | 0.099 | 0.1045 |
| 45 | 0.121 | 0.005 | 0.106 | 0.11 |
| 60 | 0.123 | 0.0045 | 0.1095 | 0.113 |
| 90 | 0.127 | 0.004 | 0.1135 | 0.111 |
| 180 | 0.129 | 0.004 | 0.1125 | 0.064 |

All publications and patents cited in this specification are herein incorporated by reference as if each individual publication or patent were specifically and individually indicated to be incorporated by reference. Although the foregoing invention has been described in some detail by way of illustration and example for purposes of clarity or understanding, it will be readily apparent to those of ordinary skill in the art in light of the teachings of this invention that certain changes and modifications may be made thereto without departing from the spirit or scope of the appended claims.

### Example 9

A formulation is provided in Table 8 for exemplary granules and tablets comprising 375 mg of API, i.e. a co-crystal of VX-950. A granulation solution is prepared by dissolving 9.38 g of SLS in 187.49 g of water in a glass beaker using a magnetic stirrer. After the SLS is dissolved, 3.13 g of HPMC E50 grade is added and the granulation liquid is mixed until all HPMC is dissolved. 105.60 g of the co-crystal and 9.60 g of Avicel PH101 and 2.40 g of croscarmellose sodium are weighed out and placed in the 1.7 L granulation bowl of the Procept Mi-Pro high shear granulator. The impeller speed is set at 800 RPM and the chopper speed is set at 1100 RPM. A syringe pump is used to control the addition rate of the granulation solution. The pump rate is set at 6.0 ml per minute. The fluid is added to the bowl using a two fluid atomization nozzle with a 1.5 mm end cap. The atomization air flow rate is set at 3.0 liter per minute. The granulator is turned on and the pump is started a few seconds later. The granulation solution is added for 10 minutes at which point the pump and impeller and chopper are stopped. The wet granules are placed in a Pyrex tray and the tray is placed in a vacuum oven overnight at 43° C. with a slight nitrogen purge. The dried granules are milled using a cone mill with a 32R screen and round impeller at 3560 RPM to reduce the granule size. 39.33 g of the milled granules are blended with 5.50 g of Lactose monohydrate, 5.50 g of Avicel PH102, 1.65 g of croscarmellose sodium, and 0.28 g of colloidal silica in a 0.5 qt v-shell blender for 96 revolutions. 2.75 g of sodium stearyl fumarate is added and the mixture is blended an additional 72 revolutions. This blend is compressed into 723 mg tablets using capsule shaped tooling using a MTS load frame at 5.9 kN compression force. The hardness of these tablets is approximately 12.5 kP. Table 9 shows a granulate composition and a tablet dosage form formulation from the granulation composition.

TABLE 8
Aqueous-Based High Shear Granulation Composition

| Component | Actual % wt. |
|---|---|
| Active ingredient VX-950 | 87.40 |
| Co-crystal | |
| MCC Avicel PH101 | 8 |
| Lactose Monohydrate | 0 |
| Croscarmellose Sodium | 2 |
| HPMC E50 | 0.70 |
| Sodium Lauryl Sulfate | 2 |

The content of water for the aqueous-based granulation above and the granulations herein can be in a range of 1 wt. % and 50 wt. % (e.g., 1, 2, 3, 4, 5, 10, 15, 17, 20, 25, 30, 35, 40, 45, 50) of the total weight of the aqueous-based granulation.

TABLE 9
Tablet Composition

| | Amount/tablet | % |
|---|---|---|
| Component | (mg) | w/w |
| VX-950 Co-crystal | 516.94 | 71.51 |
| granules* | | |
| Fast-Flo Lactose 316 | 72.30 | 10.00 |
| Micro-crystalline cellulose | 72.30 | 10.00 |
| PH102 | | |
| Croscarmellose Sodium | 21.70 | 3.00 |
| Colloidal Silica M- | 3.60 | 0.50 |
| 5P(Cabosil) | | |
| Sodium Stearyl Fumarate | 36.10 | 5.00 |
| (SSF) | | |
| TOTAL | 722.94 | 100.00 |

*Granules contain 87.4% CoX, 8% MCC PH101, 2% CCS, 2% SLS and 0.7% HPMC E50.

1. A pharmaceutical formulation comprising:
a co-crystal, the co-crystal comprising:
a) a co-former; and
b) an active pharmaceutical ingredient (API) with solubility in water is less than one part by weight of the API in ten parts by weight of water; and
a polymer,
wherein:
a weight ratio of the co-crystal to the polymer is about 0.5:99.5 to about 99.5:0.5; and
the kinetic solubility of the co-crystal after being in contact with an environment of use is at a therapeutically acceptable level for a prolonged period of time.

2. (canceled)

3. The formulation of claim 2, further comprising one or more excipients, wherein the one or more excipients comprises one or more selected from the group consisting of: a filler, a surfactant, a glidant, a lubricant and a disintegrant.

4. (canceled)

5. The formulation of claim 3, wherein the one or more excipients comprises about 5 wt. % to about 60 wt. % of the one or more fillers.

6. The formulation of claim 5, wherein the one or more fillers is selected from one or more from the group consisting of the following: mannitol, lactose, sucrose, dextrose, maltodextrin, sorbitol, xylitol, powdered cellulose, microcrystalline cellulose, silicified microcrystalline cellulose, methylcellulose, ethylcellulose, hydroxyethylcellulose, methylhydroxyethylcellulose, talc, starch, pregelatinized starch, dibasic calcium phosphate, calcium sulfate and calcium carbonate.

7-10. (canceled)

11. The formulation of claim 3, wherein the one or more excipients comprises about 1 wt. % to about 30 wt. % of the one or more disintegrants.

12. The formulation of claim 11, wherein the one or more disintegrants is selected from one or more from the group consisting of the following: croscarmellose sodium, sodium alginate, calcium alginate, alginic acid, starch, pregelatinized starch, sodium starch glycolate, crospovidone, cellulose and its derivatives, carboxymethylcellulose calcium, carboxymethylcellulose sodium, soy polysaccharide, guar gum, an ion exchange resin, an effervescent system based on food acids and an alkaline carbonate component, and sodium bicarbonate.

13. (canceled)

14. (canceled)

15. The formulation of claim 3, wherein the one or more excipients comprises about 0.1 wt. % to 30 wt. % of the one or more surfactants.

16. The formulation of claim 15, wherein the one or more surfactants is selected from one or more from the group consisting of the following: sodium lauryl sulfate, docusate sodium, polyoxyethylene sorbitan fatty acid esters, polyoxyethylene 20 stearyl ethers, polyoxyethylene alkyl ethers, polyoxyethylene castor oil derivatives, pegylated hydrogenated castor oils, sorbitan esters of fatty acids, Vitamin E or tocol derivatives, vitamin E TPGS, tocopheryl esters, lecithin, phospholipids and their derivatives, poloxamers, stearic acid, oleic acid, oleic alcohol, cetyl alcohol, mono and diglycerides, propylene glycol esters of fatty acids, glycerol esters of fatty acids, ethylene glycol palmitostearate, polyoxylglycerides, propylene glycol monocaprylate, propylene glycol monolaurate and polyglyceryl oleate.

17. (canceled)

18. (canceled)

19. The formulation of claim 3, wherein the one or more excipients comprises about 0.1 wt. % to 30 wt. % of the one or more lubricants.

20. The formulation of claim 19, wherein the one or more lubricants is selected from one or more from the group consisting of the following: talc, fatty acid, stearic acid, magnesium stearate, calcium stearate, sodium stearate, glyceryl monostearate, sodium lauryl sulfate, sodium stearyl fumarate, hydrogenated oils, fatty alcohol, fatty acid ester, glyceryl behenate, mineral oil, vegetable oil, leucine, sodium benzoate, or a combination thereof.

21. (canceled)

22. The formulation of claim 1, wherein the polymer is selected from the following: hydroxypropylmethyl cellulose (HPMC), hydroxypropylmethyl cellulose acetate succinate (HPMCAS), polyvinylpyrrolidone (PVP), polyethylene glycol (PEG), poly(ethylene oxide) (PEO), Polyvinylpyrrolidone-co-vinylacetate (PVPVA), poly(meth)acrylate (e.g., Eudragit® polymers), Polyvinylpyrrolidone (PVP), Polyvinylacetate (PVA) or a combination thereof.

23. (canceled)

24. (canceled)

25. The formulation of claim 1 comprising:
about 5 wt. % to 90 wt. % of the co-crystal;
about 5 wt. % to 90 wt. % of a filler;
about 1 wt. % to 30 wt. % of the polymer;
about 1 wt. % to 30 wt. % of a disintegrant;
about 0.1 wt. % to 30 wt. % of a surfactant; and
about 0.1 wt. % to 30 wt. % of a lubricant.

26. The formulation of claim 1 comprising:
about 20 wt. % to 75 wt. % of the co-crystal;
about 20 wt. % to 75 wt. % of a filler;
about 1 wt. % to 10 wt. % of the polymer;
about 1 wt. % to 10 wt. % of a disintegrant;
about 1 wt. % to 10 wt. % of a surfactant; and
about 1 wt. % to 10 wt. % of a lubricant.

27. The formulation of claim 1 further comprising one or more excipients, wherein the one or more excipients comprises silicifed microcrystalline cellulose, croscarmellose sodium, sodium lauryl sulfate, and sodium stearyl fumarate.

28. The formulation of claim 1, wherein the formulation is in a form of a capsule, tablet, pill, powder, granule, aqueous suspension or solution.

29-31. (canceled)

32. The formulation of claim 1, wherein the co-former is a carboxylic acid.

33. The formulation of claim 32, wherein the co-former is 4-hyrdoxybenzoic acid.

34. The formulation of claim 1, wherein the API is a protease inhibitor.

35. The formulation of claim 1, wherein the solubility of the API in water is less than one part by weight of the API in fifty parts by weight of water.

36. (canceled)

37. The formulation of claim 1, wherein the API is VX-950.

38. The formulation of claim 1, wherein the formulation is fluid-bed granulated.

39. A method of formulating a co-crystal comprising:
1) at least partially dissolving an aqueous component in an aqueous based solution, wherein the aqueous component includes:
a) a surfactant; and
b) optionally, a polymer;
2) contacting the aqueous-based solution with a co-crystal component, wherein the co-crystal component includes:
a) a co-crystal, wherein the co-crystal comprising
i) a co-former; and
ii) an active pharmaceutical ingredient (API) with solubility in water is less than one part by weight of the API in ten parts by weight of water;
b) a filler; and
c) optionally, a polymer,
provided that one of the co-crystal component and the aqueous component includes the polymer; and
3) obtaining a substantially dry blend comprising the co-crystal component and the aqueous component,
wherein:
the formulation comprising particles of the co-crystal and the polymer in intimate association with each other.

40-42. (canceled)

43. An aqueous-based granulation for formulating a drug dosage form, comprising:
a co-crystal, the co-crystal comprising:
a) a co-former; and
b) an active pharmaceutical ingredient (API)
a polymer;
wherein:
the co-crystal comprises at least 5% of the total weight of a substantially dry mass of the aqueous-based granulation;
at least 1% of the total weight of the aqueous-based granulation is water; and
the co-former and the API of the co-crystal do not dissociate when come in contact with water.

44-85. (canceled)

86. A drug dosage form comprising the substantially the aqueous-based granulation of claim 43, wherein the drug dosage form is substantially free of water.

87. A drug dosage form comprising the aqueous-based granulation of claim 86, wherein the dosage form is in a form of a capsule, tablet, pill, powder, or granule.

88-90. (canceled)

91. The drug dosage form of claim 86, wherein the API comprises at least 50% of the total weight of the dosage form.

92. A method of formulating a drug dosage form of a co-crystal comprising:
1) at least partially dissolving the co-crystal in an aqueous-based solution, wherein the aqueous component includes a polymer;
2) contacting the aqueous-based solution with a co-crystal component, wherein the co-crystal component includes:
a) a co-crystal, wherein the co-crystal comprising
i) a co-former; and
ii) an API,
wherein:
the co-crystal comprises at least 5% of the total weight of a substantially dry mass of the aqueous-based granulation;
at least 1% of the total weight of the aqueous-based granulation is water; and
the co-former and the API of the co-crystal do not dissociate when come in contact with water.

93-99. (canceled)
